(19) United States
(12) Patent Application Publication
(10) Pub. No.: US 2004/0077640 A1
(43) Pub. Date: Apr. 22, 2004

(54) Sodium channel modulators derived from 2-piperidylimidazoles

(76) Inventors: Dennis Bigg, Gif-Sur-Yvette (FR); Jacques Pommier, Paris (FR); Anne-Marie Liberatore, Auffargis (FR)

Correspondence Address: MUSERLIAN AND LUCAS AND MERCANTI, LLP, 475 PARK AVENUE SOUTH, NEW YORK, NY, 10016, US

(21) Appl. No.: 10/450,215

(22) PCT Filed: Dec. 27, 2001
(86) PCT No.: PCT/FR01/04209

(30) Foreign Application Priority Data
Dec. 28, 2000 (FR) 00/17149

(51) Int. Cl. A61K 31/541; A61K 31/496; A61K 31/5377; A61K 31/454

(52) U.S. Cl. 514/227.8; 514/235.8; 514/253.09; 514/326

(57) ABSTRACT

The invention concerns novel sodium channel modulators derived from 2-piperidylimidazoles. The inventive compounds correspond to the general formula (I), wherein: R¹ represents a hydrogen atom or an —X—Y—R⁴ radical wherein —X—Y represents a bond, a —CO—, —CO—O——CO—NH— or —CS—NH— radical and R⁴ represents an alkyl, haloalkyl, cycloalkyl, cycloalkylalkyl, aryl or aralkyl radical, said radicals being optionally substituted; R² represents in particular an alkyl, cycloalkyl, cycloalkylalkyl or aryl radical optionally substituted; and R³ represents a hydrogen atom or an alkyl, cycloalkyl, cycloalkylalkyl, or aryl radical optionally substituted.

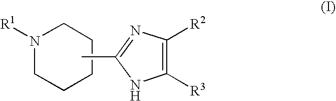

The present invention relates to new modulators of sodium channels derived from 2-piperidylimidazoles.

The compounds which modulate sodium channels are very useful for therapeutic uses such as:

the treatment or prevention of pain, and in particular:

neuropathic pains such as trigeminal neuralgia, post-herpetic pain, diabetic neuropathies, glossopharyngial neuralgias, secondary radiculopathies and neuropathies associated with metastatic infiltrations, adiposis dolorosa and pains associated with burns,

migraine,

post-operative pains,

central pains as a result of vascular cerebral incidents, thalamic lesions and multiple sclerosis, and

chronic inflammatory pains or pains linked to cancer;

the treatment of epilepsy;

the treatment of disorders of the cardiac rhythm;

the treatment of disorders linked to neurodegeneration, and in particular:

vascular cerebral incidents,

cerebral trauma, and

neurodegenerative diseases such as Alzheimer's disease, Parkinson's disease and amyotrophic lateral sclerosis;

the treatment of depression and bipolar disorders;

the treatment of irritable bowel syndrome;

the treatment of diabetic retinopathies.

The Applicant had already described, in a prior Application unpublished at the date of the present Application, compounds modulating sodium channels corresponding to general formula

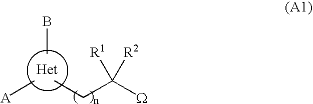

in racemic form, enantiomeric form or any combination of these forms, in which Het is a heterocycle with 5 members comprising 2 heteroatoms and such that general formula (A1) corresponds exclusively to one of the following sub-formulae:

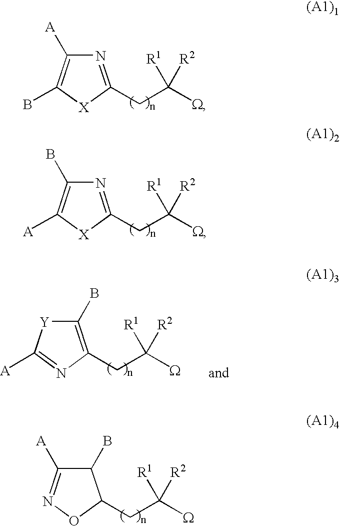

in which

A represents in particular an optionally substituted alkyl, cycloalkyl or phenyl radical;

B represents in particular a hydrogen atom or an optionally substituted alkyl, cycloalkyl or phenyl radical;

X represents in particular NH or S;

Y represents O or S;

R¹ and R² represent in particular independently a hydrogen atom, an alkyl or cycloalkyl radical;

Ω represents one of the NR⁴⁶R⁴⁷ or OR⁴⁸ radicals in which R⁴⁶ and R⁴⁷ represent in particular a hydrogen atom, an alkyl, cycloalkyl, cycloalkylalkyl or aralkyl radical or also a —COOR⁵¹ radical in which R⁵¹ represents in particular an alkyl, aryl or aralkyl radical.

A certain number of patents or patent applications describe moreover derivatives of 2-piperidylimidazoles.

The PCT Patent Application WO 96/16040 describes the compounds of general formula (A2)

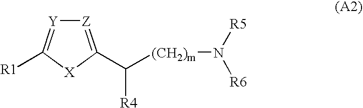

in which

R¹ represents one of the aryl, heteroaryl, aralkyl or cycloalkyl radicals optionally substituted by one to three substituents chosen independently from a halogen atom, the

CF₃, CN, OH, alkyl or alkoxy, SO₂R9 radical with R9 representing NH₂ or NHCH₃;

X represents NR2, R2 representing H or alkyl;

Y represents N or CR3;

Z represents CR3 or N;

provided however that Y and Z are not both CR3 or N at the same time;

R3 represents H, alkyl, halogen, hydroxyalkyl or phenyl optionally substituted by 1 to 3 substituents chosen from H, CF₃, CN, SO₂NH₂, OH, alkyl or alkoxy;

m represents 0, 1 or 2;

R4 represents H or alkyl;

when Z represents CR3, then R3 and R4 can also represent together —(CH₂)ₙ₁— with n1 an integer from 2 to 4 or R2 and R4 can also represent together —(CH₂)ₙ₂— with n2 an integer from 2 to 4;

R5 and R6 represent independently H, alkyl, alkoxy, aryl or aralkyl;

R4 and R5 also being able to represent together —(CH₂)ₙ₃—with n3 representing 2 or 3;

NR5R6 also being able to represent together (in particular):

the optionally substituted 2—(1,2,3,4-tetrahydroquinolyl) radical,

-a

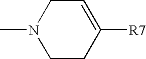

radical in which R7 represents one of the phenyl, benzyl or phenethyl radicals in which the phenyl ring can be substituted;

-a

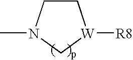

radical in which p is an integer from 1 to 3,

W is N and R8 represents H, CF₃, one of the phenyl, pyridyl or pyrimidinyl radicals optionally substituted once or twice by radicals chosen from halogen, OH, alkyl or alkoxy, or

W is CH and R8 represents phenyl optionally substituted or aralkyl optionally substituted on the aryl group;

these compounds being partial agonists or antagonists of the sub-receptors of cerebral dopamine or of the prodrug forms of such partial agonists or antagonists. Useful properties would thus be obtained in the diagnosis and treatment of affective disorders such as schizophrenia and depression as well as certain movement disorders such as Parkinson's disease.

The American Patents U.S. Pat. Nos. 5,717,100 and 6,083,349 and the PCT Applications WO 97/12876, WO 97/36587 and WO 97/47618 mainly indicate that the compounds of general formula (A3)

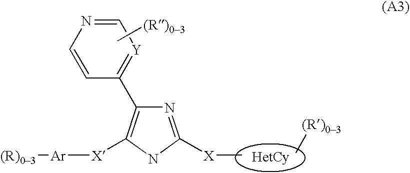

in which:

Ar represents an aryl radical optionally substituted by one to three substituents chosen independently from the R substituents;

X and X′ represent in particular a —(CH₂)ₘ—Y—(CH₂)ₙ—radical in which m and n are integers from 0 to 4 and Y represents in particular a bond, O, S, SO, SO₂, OCO, COONH or CONH;

Y represents CH or N;

HetCy represents a non aromatic heterocycle with 4 to 10 members comprising at least one nitrogen atom;

the R and R″ radicals, each time they are involved, are chosen independently from the group comprising in particular a halogen atom, an OH, CF₃, SH, NH₂ or NO₂ group and an optionally substituted alkyl, heterocyclyl or heteroaryl radical; and the R′ radical is, each time it is involved, chosen independently from the group comprising in particular the hydroxy, amino, optionally substituted alkyl, heterocyclyl and heteroaryl radicals;

can be used as anti-cancerous agents.

Apart from the aforementioned documents, the PCT Patent Application WO 00/57877 describes in particular the compounds of general formula (A4)

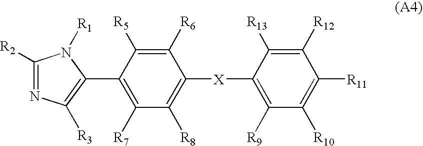

in which

R¹ represents (in particular) a hydrogen atom or an alkyl radical,

R₂ and R₃ represent independently a hydrogen atom or an alkyl, cycloalkyl, alkenyl, alkynyl, haloalkyl, aryl, aminoalkyl, hydroxyalkyl, alkoxyalkyl, alkylthio, alkylsulphinyl, alkylsulphonyl, carboxyalkyl, cyano, amino, alkylamino, aminocarbonyl, alkylaminocarbonyl, arylaminocarbonyl, aralkylaminocarbonyl, alkylcarbonylamino, arylcarbonylamino, aralkylcarbonylamino, alkylcarbonyl, heterocyclocarbonyl, aminosulphonyl, alkylaminosulphonyl and heterocyclosulphonyl radical,

X represents O, S or an NR₁₅ radical in which R₁₅ represents a hydrogen atom, an alkyl or cycloalkyl radical,

and R₅, R₆, R₇, R₈, R₉, R₁₀, R₁₁, R₁₂ and R₁₃ represent (in particular) independently a hydrogen atom, a halogen atom and an alkyl, hydroxy or alkoxy radical, as inhibitors of the sodium channels.

The Applicant has just discovered a new class of compounds modulating the sodium channels, namely the 2-piperidylimidazole derivatives described hereafter.

According to the invention, the compounds corresponding to general formula (I)

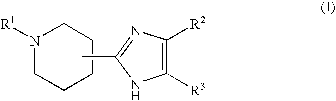

in racemic or enantiomeric form or any combination of these forms, in which:

R¹ represents a hydrogen atom or an —X—Y—R⁴ radical in which —X—Y— represents a bond, a —CO—, —CO—O—, —CO—NH— or —CS—NH— radical and R⁴ represents an alkyl, haloalkyl, cycloalkyl, cycloalkylalkyl, aryl or aralkyl radical, said aralkyl or aryl radicals being optionally substituted 1 to 4 times (preferably 1 to 3 times and more preferentially 1 to 2 times) on the aryl radical by one or more radicals chosen from a halogen atom and an alkyl, hydroxy or alkoxy radical;

R² represents an alkyl, cycloalkyl, cycloalkylalkyl or aryl radical, said aryl being optionally substituted 1 to 4 times (preferably 1 to 3 times and more preferentially 1 to 2 times) by one or more radicals chosen from a halogen atom and an alkyl, haloalkyl, hydroxy, alkoxy, alkylthio, cyano, nitro, NR⁵R⁶, SO₂R⁷, arylamino or aryl radical, said arylamino or aryl being optionally substituted 1 to 4 times (preferably 1 to 3 times and more preferentially 1 to 2 times) on the aryl group by one or more radicals chosen from a halogen atom and an alkyl, hydroxy, alkoxy or alkylthio radical, R⁵ and R⁶ representing independently a hydrogen atom or an alkyl radical or R⁵ and R⁶ forming together with the nitrogen atom already present a heterocycle with 5 to 7 members the additional members of which are chosen from —CH₂—, —O—, —S— and —NR⁸—,

R⁷ representing independently each time it is involved an alkyl radical,

R⁸ representing independently each time it is involved a hydrogen atom or an alkyl, haloalkyl, cycloalkyl, cycloalkylalkyl, aryl or aralkyl radical, said aralkyl or aryl radicals being optionally substituted 1 to 4 times (preferably 1 to 3 times and more preferentially 1 to 2 times) on the aryl radical by one or more radicals chosen from a halogen atom and an alkyl, hydroxy or alkoxy radical,

or R² represents the

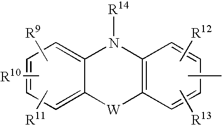

radical in which R⁹, R¹⁰, R¹¹, R¹² and R¹³ represent, independently, a hydrogen atom, a halogen, the OH group or an alkyl or alkoxy radical,

R¹⁴ represents a hydrogen atom or an alkyl radical,

and W does not exist, or represents a bond, or —O—, —S— or —NR¹⁵—, in which R¹⁵ represents a hydrogen atom or an alkyl radical,

or also R² represents the

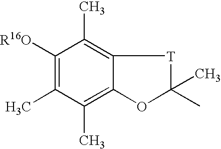

radical in which R¹⁶ represents a hydrogen atom or an alkyl radical,

and T represents a —(CH₂)ₘ— radical with m=1 or 2,

or finally R² represents the

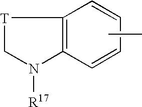

radical in which R¹⁷ represents a hydrogen atom or an alkyl, —Σ—NR¹⁸R¹⁹ or —Σ—CHR²⁰R²¹ radical,

Σ representing a linear or branched alkylene radical containing 1 to 6 carbon atoms,

R¹⁸ and R¹⁹ representing, independently, a hydrogen atom or an alkyl radical,

R²⁰ and R²¹ representing, independently, a hydrogen atom or a carbocyclic or heterocyclic aryl radical optionally substituted 1 to 4 times (preferably 1 to 3 times and more preferentially 1 to 2 times) by one or more substituents chosen independently from the alkyl, OH, halogen, nitro, alkoxy and NR²²R²³ radicals,

R²² and R²³ representing, independently, a hydrogen atom or an alkyl radical, and T represents a —(CH₂)ₘ— radical with m=1 or 2; and

R³ represents a hydrogen atom or an alkyl, cycloalkyl, cycloalkylalkyl or aryl radical, said aryl being optionally substituted 1 to 4 times (preferably 1 to 3 times and more preferentially 1 to 2 times) by one or more radicals chosen from a halogen atom and an alkyl, haloalkyl, hydroxy, alkoxy, alkylthio, cyano, nitro, NR²⁴R²⁵, SO₂R²⁶ or aryl radical optionally substituted 1 to 4 times (preferably 1 to 3 times and more preferentially 1 to 2 times) by one or more radicals chosen from a halogen atom and an alkyl, hydroxy, alkoxy or alkylthio radical,

R²⁴ and R²⁵ representing independently a hydrogen atom or an alkyl radical or R²⁴ and R²⁵ forming together with the nitrogen atom already present a heterocycle with 5 to 7 members the additional members of which are chosen from —CH₂—, —O—, —S— and —NR²,

R²⁶ representing independently each time it is involved an alkyl radical, and R²⁸ representing independently each time it is involved a hydrogen atom or an alkyl radical;

or the pharmaceutically acceptable salts of these compounds;

can be used for preparing a medicament intended to have a modulatory activity on the sodium channels.

By alkyl, unless otherwise specified, is meant a linear or branched alkyl radical containing 1 to 12 carbon atoms, and preferably 1 to 6 carbon atoms. By cycloalkyl, unless otherwise specified, is meant a monocyclic carbon system containing 3 to 7 carbon atoms. By carbocyclic or heterocyclic aryl, unless otherwise specified, is meant a carbocyclic or heterocyclic system comprising one to three condensed rings at least one of which is an aromatic ring, a system being referred to as heterocyclic when at least one of its constituent rings comprises one or more heteroatoms (O, N or S). By aryl, unless otherwise specified, is meant a carbocyclic aryl radical. By heteroaryl is meant a heterocyclic aryl radical. When the term aryl is used without further specification, there should be exclusively understood a carbocyclic aryl radical. By heterocycle is meant a condensed mono-, bi- or tricyclic system, said saturated system, partially or totally unsaturated or aromatic, being composed of rings comprising 3 to 7 members at least one of which comprises at least one heteroatom chosen from O, N and S. By haloalkyl, is meant an alkyl radical at least one of the hydrogen atoms (and optionally all) of which is replaced by a halogen atom.

By alkylthio, alkoxy, haloalkyl, cycloalkylalkyl and aralkyl radicals, is meant respectively the alkylthio, alkoxy, haloalkyl, cycloalkylalkyl and aralkyl radicals the alkyl, cycloalkyl and aryl radicals of which have the meanings indicated previously.

By heterocycle, is meant in particular the thienyl, piperidinyl, piperazinyl, quinolinyl, indolinyl and indolyl radicals. By linear or branched alkyl having 1 to 6 carbon atoms, is meant in particular the methyl, ethyl, propyl, isopropyl, butyl, isobutyl, sec-butyl and tert-butyl, pentyl, neopentyl, isopentyl, hexyl, isohexyl radicals. By carbocyclic aryl, is meant in particular the phenyl, naphthyl and phenanthryl radicals, preferably the phenyl and naphthyl radicals and more preferentially the phenyl radical. Finally, by halogen, is meant the fluorine, chlorine, bromine or iodine atoms.

By pharmaceutically acceptable salt is meant in particular addition salts with inorganic acids such as hydrochloride, hydrobromide, hydroiodide, sulphate, phosphate, diphosphate and nitrate or with organic acids such as acetate, maleate, fumarate, tartrate, succinate, citrate, lactate, methanesulphonate, p-toluenesulphonate, pamoate and stearate. The salts formed from bases such as sodium or potassium hydroxide also fall within the scope of the present invention, when they can be used. For other examples of pharmaceutically acceptable salts, reference can be made to “Salt selection for basic drugs”, Int. J. Pharm. (1986), 33, 201-217.

Preferably, the compounds of general formula (I) defined above (or their pharmaceutically acceptable salts) will have at least one of the following characteristics:

R¹ represents a hydrogen atom or an alkyl, aralkyl or cycloalkylalkyl radical or also R¹ represents an —X—Y—R⁴ radical;

R² represents a phenyl radical substituted by a halogen atom, an alkyl or alkoxy radical, an NR⁵R⁶ radical or a phenyl radical itself optionally substituted by a halogen atom or an alkoxy radical;

R³ represents a hydrogen atom or an alkyl or phenyl radical.

More preferentially, the compounds of general formula (I) defined above (or their pharmaceutically acceptable salts) will have at least one of the following characteristics:

the piperidyl ring of the compounds of general formula (I) is substituted in position 3 or 4 by the imidazolyl radical carrying the R² and R³ groups;

R¹ represents a hydrogen atom or an aralkyl or cycloalkylalkyl radical or also R¹ represents an —X—Y—R⁴ radical in which the —X—Y— group represents —CO—, —CO—O—, —CO—NH— or —CS—NH— and R⁴ represents an alkyl, haloalkyl, cycloalkyl, cycloalkylalkyl, aryl or aralkyl radical;

R² represents a phenyl radical substituted by a halogen atom, an alkyl radical or a phenyl radical itself optionally substituted by a halogen atom;

R³ represents a hydrogen atom.

Also more preferentially, the compounds of general formula (I) defined above (or their pharmaceutically acceptable salts) will have at least one of the following characteristics:

the piperidyl ring of the compounds of general formula (I) is substituted in position 3 or 4 by the imidazolyl radical carrying the R² and R³ groups;

R¹ represents a hydrogen atom or a benzyl or cyclohexylalkyl radical or also R¹ represents an —X—Y—R⁴ radical in which the —X—Y— group represents —CO—, —CO—O—, —CO—NH— or —CS—NH— and R⁴ represents an alkyl or aralkyl radical;

R² represents a phenyl radical substituted by a halogen atom, an alkyl or alkoxy radical or a phenyl radical itself optionally substituted by a halogen atom;

R³ represents a hydrogen atom.

Although the case is preferred where the piperidyl ring of the compounds of general formula (I) is substituted in position 3 or 4 by the imidazolyl radical carrying the R² and R³ groups, another useful variant is constituted by the case where the piperidyl ring of the compounds of general formula (I) is substituted in position 2 by the imidazolyl radical carrying the R² and R³. groups

According to a preferred variant of the invention, the compounds of general formula (I) defined above (or their pharmaceutically acceptable salts) are such that the R¹ radical represents an —X—Y—R⁴ radical in which:

either —X—Y— represents a bond and R⁴ represents a cycloalkyl, cycloalkylalkyl or aralkyl radical, said aralkyl radical being optionally substituted 1 to 4 times (preferably 1 to 3 times and more preferentially 1 to 2 times) on the aryl radical by a radical or radicals chosen from a halogen atom and an alkyl, hydroxy or alkoxy radical;

or —X—Y— represents a —CO—, —CO—O—, —CO—NH— or —CS—NH— radical and R⁴ represents an alkyl, haloalkyl, cycloalkyl, cycloalkylalkyl, aryl or aralkyl radical, said aralkyl or aryl radicals being optionally substituted 1 to 4 times (preferably 1 to 3 times and more preferentially 1 to 2 times) on the aryl radical by a radical or radicals chosen from a halogen atom and an alkyl, hydroxy or alkoxy radical.

According to another preferred variant of the invention, the compounds of general formula (I) defined above (or their pharmaceutically acceptable salts) are such that the R² radical represents a phenyl radical substituted by a phenyl radical itself optionally substituted by a halogen atom or an alkoxy radical.

According to yet another preferred variant of the invention, the compounds of general formula (I) in which at least one of R¹, R² and R³ is a group which traps free radicals (or their pharmaceutically acceptable salts) can be used for preparing a medicament specially intended to modulate the sodium channels and to trap the reactive oxygen species (or ROS). For this preferred variant, the same preferences as those indicated previously in general for the compounds of general formula (I) are applicable mutatis mutandis.

The compounds described (sometimes in the form of salts) in Examples 1 to 26 are particularly preferred for use according to the invention. Even more preferentially, the compounds of the invention are chosen from the compounds of Examples 1 to 9, 12 to 14, 17, 19 to 24 and 26 described hereafter.

The invention relates moreover to the use of a compound of general formula (I) as defined previously or a pharmaceutically acceptable salt of such a compound for preparing a medicament intended to have a modulatory activity on the sodium channels in the patient to which it has been administered. It is particularly preferable to use a compound of general formula (I) as defined previously or a pharmaceutically acceptable salt of such a compound for preparing a medicament intended to treat pain, in particular neuropathic pains and migraine.

A subject of the invention is also, as medicaments, the compounds of general formula (I) corresponding to general sub-formula (I)_{M}.

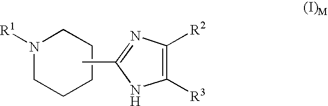

in racemic or enantiomeric form or any combination of these forms, in which:

R¹ represents a hydrogen atom or an —X—Y—R⁴ radical in which —X—Y— represents a bond, a —CO—, —CO—O—, —CO—NH— or —CS—NH— radical and R⁴ represents an alkyl, haloalkyl, cycloalkyl, cycloalkylalkyl, aryl or aralkyl radical, said aralkyl or aryl radicals being optionally substituted 1 to 4 times (preferably 1 to 3 times and more preferentially 1 to 2 times) on the aryl radical by one or more radicals chosen from a halogen atom and an alkyl, hydroxy or alkoxy radical;

R¹ represents an alkyl, cycloalkyl, cycloalkylalkyl, or aryl radical, said aryl being optionally substituted 1 to 4 times (preferably 1 to 3 times and more preferentially 1 to 2 times) by a radical or radicals chosen from a halogen atom and an alkyl, haloalkyl, hydroxy, alkoxy, alkylthio, cyano, nitro, NR⁵R⁶, SO₂R⁷, arylamino or aryl radical, said arylamino or aryl being optionally substituted 1 to 4 times (preferably 1 to 3 times and more preferentially 1 to 2 times) on the aryl group by a radical or radicals chosen from a halogen atom and an alkyl, hydroxy, alkoxy or alkylthio radical,

R⁵ and R⁶ representing independently a hydrogen atom or an alkyl radical or R⁵ and R⁶ forming together with the nitrogen atom already present a heterocycle with 5 to 7 members the additional members of which are chosen from —CH₂—, —O—, —S— and —NR⁸—,

R⁷ representing independently each time it is involved an alkyl radical,

R⁸ representing independently each time it is involved a hydrogen atom or an alkyl, haloalkyl, cycloalkyl, cycloalkylallyl, aryl or aralkyl radical, said aralkyl or aryl radicals being optionally substituted 1 to 4 times (preferably 1 to 3 times and more preferentially 1 to 2 times) on the aryl radical by a radical or radicals chosen from a halogen atom and an alkyl, hydroxy or alkoxy radical,

or R² represents the

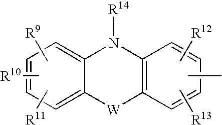

radical in which R⁹, R¹⁰, R¹¹, R¹² and R¹³ represent, independently, a hydrogen atom, a halogen, the OH group or an alkyl or alkoxy radical,

R¹⁴ represents a hydrogen atom or an alkyl radical,

and W does not exist, or represents a bond, or —O—, —S— or —NR¹⁵—, in which R¹⁵ represents a hydrogen atom or an alkyl radical,

or also R² represents the

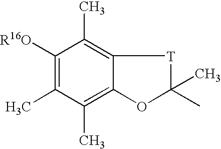

radical in which R¹⁶ represents a hydrogen atom or an alkyl radical,

and T represents a —(CH₂)ₘ— radical with m=1 or 2,

or finally R² represents the

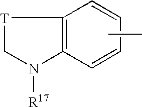

radical in which R¹⁷ represents a hydrogen atom or an alkyl, —Σ—NR¹⁸R¹⁹ or —Σ—CHR²⁰R²¹ radical,

Σ representing a linear or branched alkylene radical containing 1 to 6 carbon atoms,

R¹⁸ and R¹⁹ representing, independently, a hydrogen atom or an alkyl radical,

R²⁰ and R²¹ representing, independently, a hydrogen atom or a carbocyclic or heterocyclic aryl radical optionally substituted 1 to 4 times (preferably 1 to 3 times and more preferentially 1 to 2 times) by one or more substituents chosen independently from the alkyl, OH, halogen, nitro, alkoxy and NR²²R²³ radicals,

R²² and R²³ representing, independently, a hydrogen atom or an alkyl radical,

and T represents a —(CH₂)ₘ— radical with m=1 or 2; and

R³ represents a hydrogen atom or an alkyl, cycloalkyl, cycloalkylalkyl, or aryl radical, said aryl being optionally substituted 1 to 4 times (preferably 1 to 3 times and more preferentially 1 to 2 times) by a radical or radicals chosen from a halogen atom and an alkyl, haloalkyl, hydroxy, alkoxy, alkylthio, cyano, nitro, NR²⁴R²⁵, SO₂R²⁶ or aryl radical optionally substituted 1 to 4 times (preferably 1 to 3 times and more preferentially 1 to 2 times) by a radical or radicals chosen from a halogen atom and an alkyl, hydroxy, alkoxy or alkylthio radical,

R²⁴ and R²⁵ representing independently a hydrogen atom or an alkyl radical or R²⁴ and R²⁵ forming together with the nitrogen atom already present a heterocycle with 5 to 7 members the additional members of which are chosen from —CH₂—, —O—, —S— and —NR²⁸—,

R²⁶ representing independently each time it is involved an alkyl radical, and R²⁸ representing independently each time it is involved a hydrogen atom or an alkyl radical;

it being understood however that when R² does not represent one of the

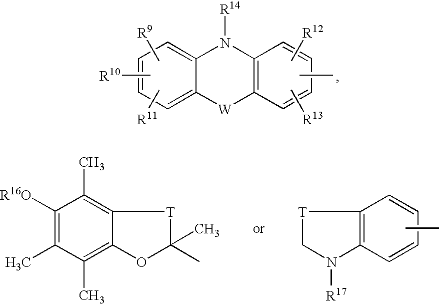

radicals,

then R¹ does not represent a radical chosen from a hydrogen atom, an alkyl radical, an alkoxy radical or an optionally substituted aryl radical;

or the pharmaceutically acceptable salts of the compounds of general formula (I)_{M}.

A subject of the invention is also, as medicaments, the compounds described (sometimes in the form of salts) in Examples 1 to 19 and their pharmaceutically acceptable salts.

A subject of the invention is also, as new products, the compounds of general formula (I)_{M} as defined previously or their salts. It also relates, as new products, to the compounds described in Examples 1 to 26 (sometimes in the form of salts) and their salts.

The invention also relates to the pharmaceutical compositions comprising, as active ingredient, at least one compound of general formula (I)_{M} as defined previously or a pharmaceutically acceptable salt of such a compound. A subject of the invention is also the pharmaceutical compositions comprising, as active ingredient, at least one compound described in Examples 1 to 26 (sometimes in the form of salts) or a pharmaceutically acceptable salt of such compound.

As regards the aforementioned products, medicaments and pharmaceutical compositions, the preferences described for the compounds of general formula (I) are applied mutatis mutandis.

The pharmaceutical compositions containing a compound of the invention can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions containing a compound of the invention can also be presented in liquid form, for example, solutions, emulsions, suspensions or syrups. Appropriate liquid supports can be, for example, water, organic solvents such as glycerol or glycols, as well as their mixtures, in varying proportions, in water.

The administration of a medicament according to the invention can be carried out by topical, oral, parenteral route, by intramuscular injection, etc.

The administration dose envisaged for a medicament according to the invention is comprised between 0.1 mg and 10 g according to the type of active compound used.

According to the invention, the compounds of general formula (I) can be prepared by the processes described hereafter.

PREPARATION OF THE COMPOUNDS OF THE INVENTION:

Two synthesis routes can be used to prepare the compounds of general formula (I), in which R¹, R² and R³ have the meaning indicated previously. These synthesis routes are summarized in Diagram 1 below. According to the labile or non-labile nature of the R¹ radical, a person skilled in the art will choose one or other of the synthesis routes.

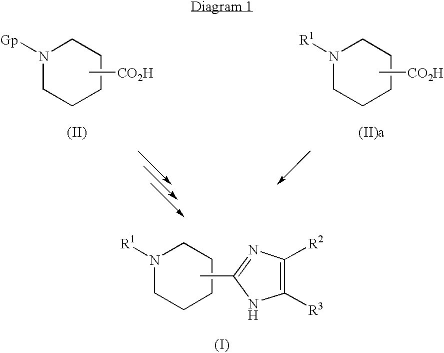

ROUTE 1: Synthesis from Compounds of General Formula (II):

The compounds of general formula (I), in which R¹, R² and R³ have the meaning indicated previously, can be prepared by the general synthesis route illustrated below (Diagram 2) starting from the intermediates of general formula (II) in which Gp is a protective group for an amine function (for example a protective group of carbamate type or any other protective group that a person skilled in the art considers appropriate, and in particular those mentioned in: Protective groups in organic synthesis, 2nd ed., (John Wiley & Sons Inc., 1991)). The acid of general formula (II) is condensed with the α-halogenoketone of general formula (III) then the protected compound of general formula (IV) is deprotected in order to produce the compound of general formula (V) before being appropriately functionalized in order to produce the compound of general formula (I) (the last two stages being unnecessary in the particular case where R¹=Gp).

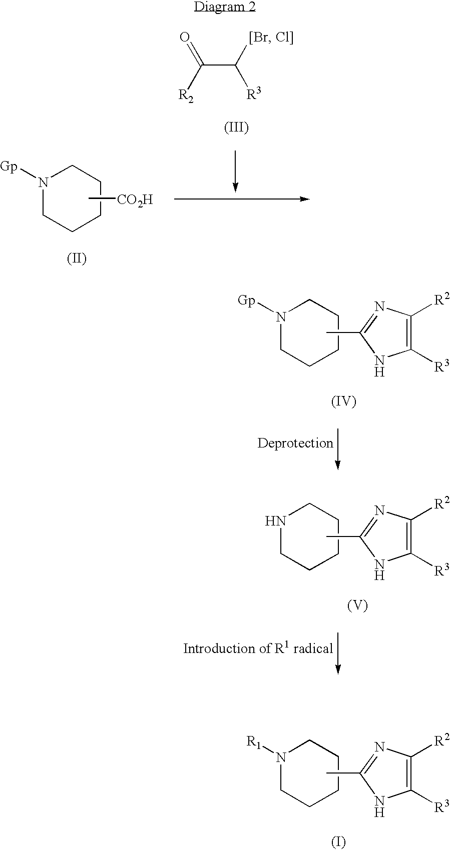

Preparation of Compounds of General Formula (II)

The acids of general formula (II) are commercially available or can be easily obtained from commercially available compounds according to standard organic synthesis methods well known to a person skilled in the art.

Preparation of Compounds of General Formula (III)

The α-halogenoketones of general formula (III) can be easily prepared from the corresponding ketones of general formula (III.i) by a halogenation reaction (Diagram 3) known to a person skilled in the art.

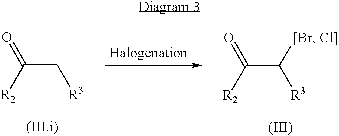

For example, in the case of bromination, the ketone of general formula (III.i) can be converted to the corresponding α-bromoketone by reaction with a bromination agent such as CuBr₂ (J. Org. Chem. (1964), 29, 3459), bromine (J. Het. Chem. (1988), 25, 337), N-bromosuccinimide (J. Amer. Chem. Soc. (1980), 102, 2838) in the presence of acetic acid in a solvent such as ethyl acetate or dichloromethane, HBr or Br₂ in ether or acetic acid (Biorg. Med. Chem. Lett. (1996), 6(3), 253-258; J. Med. Chem. (1988), 31(10), 1910-1918) or also using a bromination resin (J. Macromol. Sci. Chem. (1977), A11, (3) 507-514).

Preparation of Compounds of General Formula (IV) from Compounds of General Formula (II)

The compounds of general formula (IV) can be obtained from the acids of general formula (II) condensed according to methods known to a person skilled in the art, and for example by adding cesium carbonate followed by condensation with an α-halogenoketone of general formula (III) followed by the addition of a large excess of ammonium acetate (for example 15 or 20 equivalents per equivalent of acid of general formula (II)). This reaction is preferably carried out in a mixture of xylenes and while heating (it is also possible, if appropriate, to simultaneously eliminate the water formed during the reaction).

Preparation of Compounds of General Formula (V) from Compounds of General Formula (IV)

The deprotection for releasing the amine function of the piperidine is carried out according to standard methods known to a person skilled in the art who can in particular refer to the following publication: Protective groups in organic synthesis, 2nd ed., (John Wiley & Sons Inc., 1991).

Preparation of the Compounds of General Formula (I) from the Compounds of General Formula (V)

According to the type of R¹ radical to be introduced into the amine function of the piperidine, the last stage of the process is chosen appropriately by a person skilled in the art, and can, for example, be carried out according to the methods described hereafter and summarized in Diagrams 4 to 8.

When R¹ is an alkyl, cycloalkylalkyl, aralkyl or haloalkyl radical, the compounds of general formula (I) can be obtained, Diagram 4, by a nucleophilic substitution reaction of the compound of general formula (VI), in which Δ represents an alkyl, cycloalkylalkyl, aralkyl or haloalkyl radical, on the amine function of the piperidyl group of the compound of general formula (V).

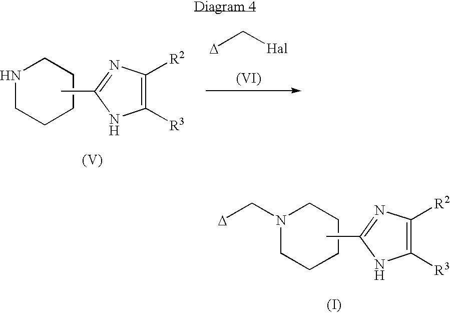

When R¹ is a cycloalkyl radical, the compounds of general formula (I) can be obtained, Diagram 5, by a condensation reaction of the compounds of general formula (V) with the cycloalkylketones of general formula (VII), in which n represents an integer from 0 to 4, in the presence of a reducing agent such as sodium triacetoxyborohydride or sodium borohydride in a lower aliphatic alcohol such as methanol and optionally in the presence of molecular sieves at ambient temperature.

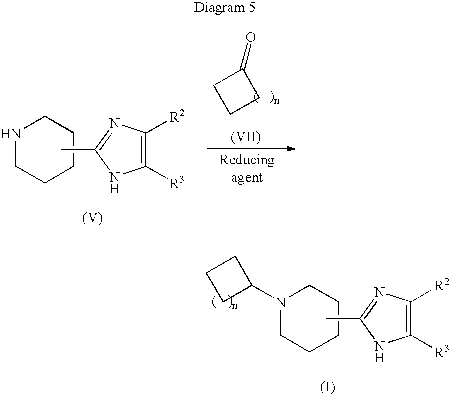

When R¹ is an alkylcarbonyl or aralkylcarbonyl radical, the compounds of general formula (I) can be obtained, Diagram 6, by condensation of the acid of general formula (VIII) (or of the corresponding acid chloride), in which Δ represents an alkyl or aralkyl radical, on the amine function of the piperidyl group of the compound of general formula (V) under standard conditions of peptide synthesis.

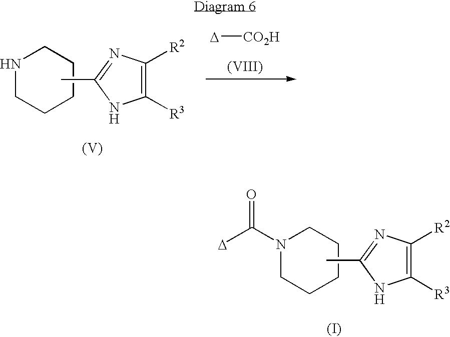

When R¹ is an alkylaminocarbonyl or aralkylaminocarbonyl radical, the compounds of general formula (I) can be obtained, Diagram 7, by condensation in an inert solvent such as dichloromethane or 1,2-dichloroethane of the isocyanate or the isothiocyanate of general formula (IX), in which Δ represents an alkyl or aralkyl radical, on the amine function of the piperidyl group of the compound of general formula (V).

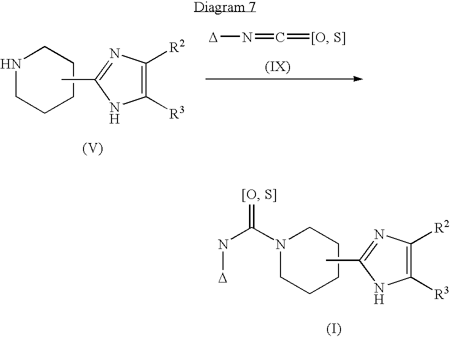

When R¹ is an alkoxycarbonyl or aralkoxycarbonyl radical, the compounds of general formula (I) can be obtained, Diagram 8, by condensation of the compound of general formula (X), in which Δ represents an alkyl or aralkyl radical, on the amine function of the piperidyl group of the compound of general formula (V). The reaction is preferably carried out in the presence of a base such as for example NaOH.

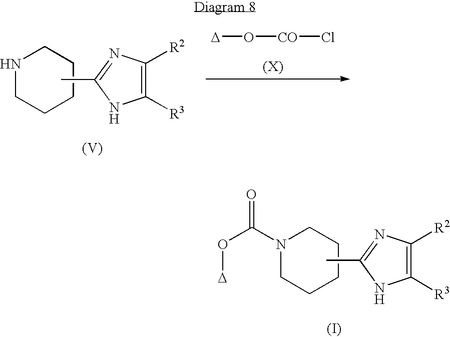

ROUTE 2: Synthesis from Compounds of General Formula (II)a:

Certain compounds of general formula (I), in which R² and R³ have the meaning indicated previously and R¹ is for example a —CO—O—R⁴ radical in which R⁴ is as defined previously, can be prepared in a single stage by the general synthesis route illustrated below (Diagram 9) starting from the compounds of general formula (II)a. This reaction is in all points similar to that already described previously between the compounds of general formula (II) and the compounds of general formula (III).

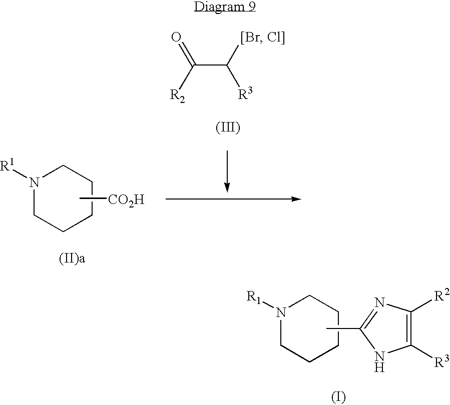

Preparation of the compounds of General Formula (II)a

The acids of general formula (II)a are commercially available or can easily be obtained from commercially available compounds according to standard organic synthesis methods well known to a person skilled in the art.

Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference.

The following examples are presented in order to illustrate the above procedures and should in no way be considered to limit the scope of the invention.

### EXAMPLES

### Example 1

butyl 4-[4-(4′-bromo-1,1′-biphenyl-4-yl)-1H-imidazol-2-yl]piperidine-1-carboxylate

1.1) 1-(butoxycarbonyl)piperidine-4-carboxylic acid

A mixture containing a 1N solution of sodium hydroxide (100 ml) and piperidine-4-carboxylic acid (12.9 g, 0.1 mol) is stirred at 23° C. for a few minutes. A 1N solution of sodium hydroxide (100 ml) and n-butyl chloroformate (13.65 g; 0.1 mol) is then added dropwise simultaneously without the temperature of the reaction medium exceeding 20° C. After stirring for 16 hours at this temperature, a 1N solution of hydrochloric acid is added until an acid pH is obtained. After extracting with ethyl acetate (twice 50 ml), washing the organic phase with a saturated solution of sodium chloride and drying over sodium sulphate, the solvents are evaporated off using a rotary evaporator. The oil obtained crystallizes from an isopropyl ether-isopentane mixture. The solid is filtered on frit and washed with isopentane. A white-coloured powder is obtained with a yield of 83%.

MH+=230.1.

1.2) 2-bromo-1-(4′-bromo-1,1′-biphenyl-4-yl)ethanone

4-(4-bromophenyl)acetophenone (10 g; 36.3 mmol) is dissolved in a methanol/dichloromethane mixture (100/150 ml). A pyridine hydrobromide perbromide polymer resin (65 g) is added and the reaction medium is stirred at 23° C. for 20 hours. The resin is recovered by filtration on frit and rinsed with dichloromethane. The filtrate obtained is evaporated to dryness and the resulting solid is rinsed with a dichloromethane-heptane solvent mixture 1-1 in small volumes. A light beige-coloured powder is obtained with a yield of 70%.

NMR H¹(δppm, DMSO): 4.96 (s, 2H); 7.69-7.75 (m, 4H); 7.90 (dd, 2H); 8.08 (dd, 2H).

1.3) butyl 4-[4-(4′-bromo-1,1′-biphenyl-4-yl)-1H-imidazol-2-yl]piperidine-1-carboxylate

A mixture containing 1-(butoxycarbonyl)piperidine-4-carboxylic acid (prepared in Stage 1.1; 2.29 g; 0.01 mol) and cesium carbonate (1.62 g, 0.005 mol) in 30 ml of anhydrous methanol is stirred for one hour. This mixture is evaporated to dryness then diluted with 40 ml of dimethylformamide. 2-bromo-1-(4′-bromo-1,1′-biphenyl-4-yl)ethanone prepared previously is added (4.00 g; 0.01 mol) then the resulting mixture is stirred for 2 hours. The solvent is evaporated off using a vane pump. 50 ml of xylenes are added and the cesium bromide is filtered on frit. Ammonium acetate (15.4 g; 0.2 mol) is then added and the mixture is heated under reflux for 1 hour 30 minutes before being poured into ice-cooled water to which 80 ml of ethyl acetate is added. After decanting, the organic phase is washed with a saturated solution of sodium chloride. The organic phase is then dried over magnesium sulphate and the solvent is evaporated off. The oil obtained crystallizes from isopropyl ether. The reaction medium is filtered on frit and recrystallized from 15 ml of isopropyl acetate. After filtering again on frit, the solid recovered is rinsed with isopropyl ether then with isopentane before drying under vacuum. A white-coloured powder is obtained with a yield of 17%. Melting point: 150-152° C.

### Example 2

tert-butyl 4-[4-(1,1′-biphenyl-4-yl)-1H-imidazol-2-yl]piperidine-1-carboxylate

The experimental protocol used is identical to that described for Stages 1.2 and 1.3 of Example 1, with Boc-isonipecotic acid (13.8 g, 0.06 mol) replacing intermediate 1.1. The final product obtained is a beige-coloured powder with a yield of 62%. Melting point: 120-122° C.

### Example 3

4-[4-(1,1′-biphenyl-4-yl)-1H-imidazol-2-yl]piperidine hydrochloride

A suspension containing the compound of Example 2 (14.9 g, 0.037 mol) in 100 ml of ethyl acetate is cooled down to approximately 5° C. 100 ml of a 3N solution of hydrochloric acid in ethyl acetate is added dropwise at this temperature. The reaction mixture is then stirred for approximately one hour at 23° C. The precipitate obtained is filtered on frit then rinsed with ethyl acetate and ether. After drying under vacuum, a beige-coloured powder is obtained with a yield of 100%. Melting point: >260° C.

### Example 4

1-benzyl-4-[4-(1,1′-biphenyl-4-yl)-1H-imidazol-2-yl]piperidine

4-[4-(1,1′-biphenyl-4-yl)-1H-imidazol-2-yl]piperidine (prepared in Example 3; 0.6 g; 0.0018 mol) is dissolved in 20 ml of acetonitrile. Triethylamine (0.6 ml; 0.0044 mol) then benzyl bromide (0.3 ml; 0.0024 mol) are added. After stirring for 1 hour at 23° C., the solvents are evaporated off. 50 ml of water and 60 ml of ethyl acetate are then added to the residue and the aqueous phase is extracted with ethyl acetate. The combined organic phases are washed with a saturated solution of sodium chloride then dried over magnesium sulphate. The solvents are evaporated off and the oil obtained is purified on a silica column (eluent: CH₂Cl₂—MeOH/95-5). A white-coloured powder is obtained with a yield of 14%. Melting point: 110-112° C.

### Example 5

1-(cyclohexylmethyl)-4-[4-(4-fluorophenyl)-1H-imidazol-2-yl]piperidine

4-[4-(4-fluorophenyl)-1H-imidazol-2-yl]piperidine (prepared according to a similar operating method to that of Example 1; 0.1 g; 0.0004 mol) is dissolved in 5 ml of methanol. Cyclohexanecarboxaldehyde (0.06 ml; 0.0005 mol) is added. After stirring for one hour at 23° C., sodium triacetoxyborohydride (0.17 g; 0.0008 mol) is added. After stirring for two days at this temperature, approximately 10 ml of water is added. The aqueous phase is extracted twice with 20 ml of ethyl acetate and the organic phase obtained is washed with a saturated solution of sodium chloride before being dried over magnesium sulphate and concentrated using a rotary evaporator. The residual oil is crystallized using ether. After filtering on frit, the crystals are washed with ether and with isopentane. A beige-coloured powder is obtained with a yield of 37%. Melting point: 220-222° C.

### Example 6

4-[4-(1,1′-biphenyl-4-yl)-1H-imidazol-2-yl]-N-butylpiperidine-1-carboxamide

4-[4-(1,1′-biphenyl-4-yl)-1H-imidazol-2-yl]piperidine (prepared in Example 3; 1.12 g; 0.003 mol) is added to 20 ml of dichloro-1,2-ethane. Triethylamine (0.84 ml; 0.006 mol) then n-butylisocyanate (0.34 ml; 0.003 mol) are added to this suspension at 23° C. After heating at approximately 50° C. for 30 minutes, the reaction mixture is stirred at 23° C. for two days. 20 ml of water is then added. The precipitate formed is filtered on frit then washed with dichloro-1,2-ethane and with ether. After drying under vacuum, a white-coloured powder is obtained with a yield of 46%. Melting point: 99-100° C.

### Example 7

4-[4-(1,1′-biphenyl-4-yl)-1H-imidazol-2-yl]-N-butylpiperidine-1-carbothioamide

4-[4-(1,1′-biphenyl-4-yl)-1H-imidazol-2-yl]piperidine (prepared in Example 3; 1.12 g; 0.003 mol) is added to 25 ml of dichloro-1,2-ethane. Triethylamine (0.84 ml; 0.006 mol) then n-butylisothiocyanate (0.38 ml; 0.003 mol) are added to this suspension at 23° C. After heating at approximately 50° C. for 30 minutes, the reaction mixture is stirred at 23° C. for two days. 20 ml of water is then added. The precipitate formed is filtered on frit then washed with dichloro-1,2-ethane and with ether. The solid obtained is purified on a silica column (eluent: CH₂Cl₂-ethanol: 90-10). A white-coloured powder is obtained with a yield of 31%. Melting point: 102-103° C.

### Example 8

ethyl 4-[4-(1,1′-biphenyl-4-yl)-1H-imidazol-2-yl]piperidine-1-carboxylate

4-[4-(1,1′-biphenyl-4-yl)-1H-imidazol-2-yl]piperidine hydrochloride (prepared in Example 3; 1.12 g; 0.003 mol) is added to 30 ml of dichloromethane. Triethylamine (0.83 ml; 0.006 mol) then n-ethyl chloroformate (0.28 ml; 0.003 mol) are added to this suspension at 23° C. The reaction mixture is stirred at 23° C. for sixteen hours. Triethylamine (0.42 ml; 0.003 mol) then 20 ml of water are then added. The aqueous phase is extracted twice with 20 ml of ethyl acetate and the organic phase obtained is washed with a saturated solution of sodium chloride before being dried over magnesium sulphate and concentrated using a rotary evaporator. The oil obtained is purified on a silica column (eluent: ethyl acetate-heptane: 95-5). A pale yellow-coloured powder is obtained with a yield of 20%. Melting point: 71-72° C.

### Example 9

4-[4-(1,1′-biphenyl-4-yl)-1H-imidazol-2-yl]-1-pentanoylpiperidine

4-[4-(1,1′-biphenyl-4-yl)-1H-imidazol-2-yl]piperidine hydrochloride (prepared in Example 3; 1.12 g; 0.003 mol) is added to 30 ml of dichloromethane. Triethylamine (0.83 ml; 0.006 mol) then pentanoyl chloride (0.36 ml; 0.003 mol) are added to this suspension at 23° C.; the reaction mixture is stirred at 23° C. for 24 hours then triethylamine (0.42 ml; 0.003 mol) is added. After stirring for one hour at 23° C., 20 ml of water is added. Once decanted, the aqueous phase is extracted twice with 20 ml of dichloromethane. The organic phases are washed with a saturated solution of sodium chloride before being dried over magnesium sulphate and concentrated using a rotary evaporator. The oil obtained is purified on a silica column (eluent: dichloromethane/ethanol: 100 to 95-5). A white-coloured powder is obtained with a yield of 15%. Melting point: 215-216° C.

The compounds of Examples 10 to 26 are obtained according to procedures similar to those described in Examples 1 to 9 or above in the section entitled “Preparation of compounds of general formula (I)”.

### Example 10

tert-butyl 4-[4-(4-fluorophenyl)-1H-imidazol-2-yl]piperidine-1-carboxylate

Free base. Melting point: 180-182° C.

### Example 11

4-[4-(4-fluorophenyl)-1H-imidazol-2-yl]piperidine

Hydrochloride. Melting point: >260° C.

### Example 12

1-benzyl4-[4-(1,1′-biphenyl-4-yl)-1H-imidazol-2-yl]piperidine

Free base. Melting point: 110-112° C.

### Example 13

butyl 4-[4-(4-tert-butylphenyl)-1H-imidazol-2-yl]piperidine-1-carboxylate

Free base. Melting point: 98-100° C.

### Example 14

1-benzyl-3-[4-(4-fluorophenyl)-1H-imidazol-2-yl]piperidine

Hydrochloride. MH+=336.2.

### Example 15

butyl 3-[4-(4-fluorophenyl)-1H-imidazol-2-yl]piperidine-1-carboxylate

Free base. Melting point: 96.5° C.

### Example 16

2-[4-(4-fluorophenyl)-1H-imidazol-2-yl]piperidine

Hydrochloride. Melting point: 222-223° C.

### Example 17

1-benzyl-2-[4-(4-fluorophenyl)-1H-imidazol-2-yl]piperidine

Free base. Melting point: 181.6° C.

### Example 18

butyl 2-[4-(4-fluorophenyl)-1H-imidazol-2-yl]piperidine-1-carboxylate

Free base. Melting point: 157.4° C.

### Example 19

4-[4-(1,1′-biphenyl-4-yl)-1H-imidazol-2-yl]-1-hexylpiperidine

Free base. Melting point: 138-139° C.

### Example 20

butyl 4-[4-(1,1′-biphenyl-4-yl)-1H-imidazol-2-yl]piperidine-1-carboxylate

Free base. Melting point: 73-74° C.

### Example 21

2-[4-(1,1′-biphenyl-4-yl)-1H-imidazol-2-yl]piperidine

Hydrochloride. MH+=304.2.

### Example 22

3-[4-(1,1′-biphenyl-4-yl)-1H-imidazol-2-yl]piperidine

Hydrochloride. Melting point: 226-227° C.

### Example 23

butyl 2-[4-(1,1′-biphenyl-4-yl)-1H-imidazol-2-yl]piperidine-1-carboxylate

Free base. Melting point: 166-167° C.

### Example 24

butyl 3-[4-(1,1′-biphenyl-4-yl)-1H-imidazol-2-yl]piperidine-1-carboxylate

Free base. Melting point: 116-117° C.

### Example 25

4-[4-(4-methoxyphenyl)-1H-imidazol-2-yl]piperidine

Hydrochloride. Melting point: 272-273° C.

### Example 26

butyl 4-[4-(4-methoxyphenyl)-1H-imidazol-2-yl]piperidine-1-carboxylate

Free base. Melting point: 48-50° C.

Pharmacological Study of the Products of the Invention

Bond Test on the Sodium Channels of the Cerebral Cortices of the Rat

The test consists in measuring the interaction of the compounds vis-à-vis the bond of tritiated batrachotoxin on the voltage-dependent sodium channels according to the protocol described by Brown (J. Neurosci. (1986), 6, 2064-2070).

Preparation of Homogenates of Cerebral Cortices of the Rat

The cerebral cortices of Sprague-Dawley rats weighing 230-250 g (Charles River, France) are removed, weighed and homogenized using a Potter grinder provided with a teflon piston (10 strokes) in 10 volumes of isolation buffer the composition of which is as follows (0.32 M sucrose, 5 mM K₂HPO₄, pH 7.4). The homogenate is subjected to a first centrifugation at 1000 g for 10 minutes. The supernatant is removed and centrifuged at 20000 g for 15 minutes. The pellet is taken up in the isolation buffer and centrifuged at 20000 g for 15 minutes. The pellet obtained is resuspended in incubation buffer (50 mM HEPES, 5.4 mM KCl, 0.8 mM MgSO₄, 5.5 mM glucose, 130 mM choline chloride pH 7.4) then aliquoted and stored at −80° C. until the day of assay. The final protein concentration is comprised between 4 and 8 mg/ml. The assay of proteins is carried out using a kit marketed by BioRad (France).

Measurement of the Bond of Tritiated Batrachotoxin

The bond reaction is carried out by incubating for 1 hour 30 minutes at 25° C. 100 μl of homogenate of rat cortex containing 75 μg of proteins with 100 μl of [³H] batrachotoxin-A 20-alpha benzoate (37.5 Ci/mmol, NEN) at 5 nM (final concentration), 200 μl of tetrodotoxin at 1 μM (final concentration) and scorpion venom at 40 μg/ml (final concentration) and 100 μl of incubation buffer alone or in the presence of the products to be tested at different concentrations. The non-specific bond is determined in the presence of 300 μM of veratridine and the value of this non-specific bond is subtracted from all the other values. The samples are then filtered using a Brandel (Gaithersburg, Md., USA) using Unifilter GF/C plates pre-incubated with 0.1% of polyethylene imine (20 μl/well) and rinsed twice with 2 ml of filtration buffer (5 mM HEPES, 1.8 mM CaCl₂, 0.8 mM MgSO₄, 130 mM choline chloride, pH 7.4). After having added 20 μl of Microscint 0®, the radioactivity is counted using a liquid scintillation counter (Topcount, Packard). The measurement is carried out in duplicate. The results are expressed as a % of the specific bond of tritiated batrachotoxin relative to the control.

Results

The compounds of Examples 1 to 9, 12 to 14, 17, 19 to 24 and 26 described above all show an IC₅₀ lower than or equal to 1 μM.

1. Use of a compound of general formula (I) 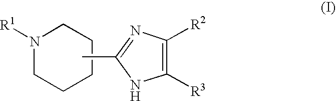
in racemic or enantiomeric form or any combination of these forms, in which:
R¹ represents a hydrogen atom or an —X—Y—R⁴ radical in which —X—Y— represents a bond, a —CO—, —CO—O—, —CO—NH— or —CS—NH— radical and R⁴ represents an alkyl, haloalkyl, cycloalkyl, cycloalkylalkyl, aryl or aralkyl radical, said aralkyl or aryl radicals being optionally substituted 1 to 4 times on the aryl radical by a radical or radicals chosen from a halogen atom and an alkyl, hydroxy or alkoxy radical;
R² represents an alkyl, cycloalkyl, cycloalkylalkyl or aryl radical, said aryl being optionally substituted 1 to 4 times by a radical or radicals chosen from a halogen atom and an alkyl, haloalkyl, hydroxy, alkoxy, alkylthio, cyano, nitro, NR⁵R⁶, SO₂R⁷, arylamino or aryl radical, said arylamino or aryl being optionally substituted 1 to 4 times on the aryl group by a radical or radicals chosen from a halogen atom and an alkyl, hydroxy, alkoxy or alkylthio radical,
R⁵ and R⁶ representing independently a hydrogen atom or an alkyl radical or R⁵ and R⁶ forming together with the nitrogen atom already present a heterocycle with 5 to 7 members the additional members of which are chosen from —CH₂—, —O—, —S— and —NR⁸—,
R⁷ representing independently each time it is involved an alkyl radical,
R⁸ representing independently each time it is involved a hydrogen atom or an alkyl, haloalkyl, cycloalkyl, cycloalkylalkyl, aryl or aralkyl radical, said aralkyl or aryl radicals being optionally substituted 1 to 4 times on the aryl radical by one or more radicals chosen from a halogen atom and an alkyl, hydroxy or alkoxy radical,
or R² represents the 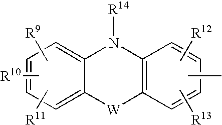
radical in which R⁹, R¹⁰, R¹¹, R¹² and R¹³ represent, independently, a hydrogen atom, a halogen, the OH group or an alkyl or alkoxy radical,
R¹⁴ represents a hydrogen atom or an alkyl radical, and W does not exist, or represents a bond, or —O—, —S— or —NR¹⁵—, in which R¹⁵ represents a hydrogen atom or an alkyl radical,
or also R² represents the 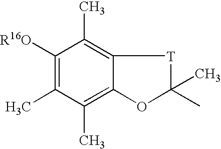
radical in which R¹⁶ represents a hydrogen atom or an alkyl radical,
and T represents a —(CH₂)ₘ— radical with m=1 or 2,
or finally R² represents the 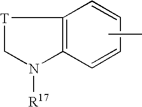
radical in which R¹⁷ represents a hydrogen atom or an alkyl, —Σ—NR¹⁸R¹⁹ or —Σ—CHR²⁰R²¹ radical,
Σ representing a linear or branched alkylene radical containing 1 to 6 carbon atoms,
R¹⁸ and R¹⁹ representing, independently, a hydrogen atom or an alkyl radical,
R²⁰ and R²¹ representing, independently, a hydrogen atom or a carbocyclic or heterocyclic aryl radical optionally substituted 1 to 4 times by one or more substituents chosen independently from the alkyl, OH, halogen, nitro, alkoxy and NR²²R²³ radicals,
R²² and R²³ representing, independently, a hydrogen atom or an alkyl radical,
and T represents a —(CH₂)ₘ— radical with m=1 or 2; and
R³ represents a hydrogen atom or an alkyl, cycloalkyl, cycloalkylalkyl or aryl radical, said aryl being optionally substituted 1 to 4 times by a radical or radicals chosen from a halogen atom and an alkyl, haloalkyl, hydroxy, alkoxy, alkylthio, cyano, nitro, NR²⁴R²⁵, SO₂R²⁶ or aryl radical optionally substituted 1 to 4 times by a radical or radicals chosen from a halogen atom and an alkyl, hydroxy, alkoxy or alkylthio radical,
R²⁴ and R²⁵ representing independently a hydrogen atom or an alkyl radical or R²⁴ and R²⁵ forming together with the nitrogen atom already present a heterocycle with 5 to 7 members the additional members of which are chosen from —CH₂—, —O—, —S— and —NR²⁸—,
R²⁶ representing independently each time it is involved an alkyl radical, and R²⁸ representing independently each time it is involved a hydrogen atom or an alkyl radical;
or a pharmaceutically acceptable salt of a compound of general formula (I);
for preparing a medicament intended to have a modulatory activity on the sodium channels.

2. Use according to claim 1, characterized in that the compound of general formula (I) or its pharmaceutically acceptable salt is such that:
R¹ represents a hydrogen atom or an alkyl, aralkyl or cycloalkylalkyl radical or also
R¹ represents a —X—Y—R⁴ radical;
R² represents a phenyl radical substituted by a halogen atom, an alkyl or alkoxy radical, an NR⁵R⁶ radical or a phenyl radical itself optionally substituted by a halogen atom or an alkoxy radical;
R³ represents a hydrogen atom or an alkyl or phenyl radical.

3. Use according to claim 1 or 2, characterized in that the compound of general formula (I) or its pharmaceutically acceptable salt is such that:
the piperidyl ring of the compounds of general formula (I) is substituted in position 3 or 4 by the imidazolyl radical carrying the R² and R³ groups;
R¹ represents a hydrogen atom or an aralkyl or cycloalkylalkyl radical or also R^{I} represents an —X—Y—R⁴ radical in which the —X—Y— group represents —CO—, —CO—O—, —CO—NH— or —CS—NH— and R⁴ represents an alkyl, haloalkyl, cycloalkyl, cycloalkylalkyl, aryl or aralkyl radical;
R² represents a phenyl radical substituted by a halogen atom, an alkyl radical or a phenyl radical itself optionally substituted by a halogen atom;
R³ represents a hydrogen atom.

4. Use according to claim 1, characterized in that the compound of general formula (I) is chosen from the following compounds:
-butyl 4-[4-(4′-bromo-1,1′-biphenyl-4-yl)-1H-imidazol-2-yl]piperidine-1-carboxylate;
-tert-butyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]piperidine;
-1-benzyl-4-[4-(1,1′-biphenyl-4-yl)-1H-imidazol-2-yl]piperidine;
-1-(cyclohexylmethyl)-4-[4-(4-fluorophenyl)-1H-imidazol-2-yl]piperidine;
-4-[4-(1,1′-biphenyl-4-yl)-1H-imidazol-2-yl]-N-butylpiperidine-1-carboxamide;
-4-[4-(1,1′-biphenyl-4-yl)-1H-imidazol-2-yl]-N-butylpiperidine-1-carbothioamide;
-ethyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]-1-pentanoylpiperidine;
-tert-butyl 4-[4-(4-fluorophenyl)-1H-imidazol-2-yl]piperidine-1-carboxylate;
-4-[4-(4-fluorophenyl)-1H-imidazol-2-yl]piperidine;
-1-benzyl-4-[4-(1,1′-biphenyl-4-yl)-1H-imidazol-2-yl]piperidine;
-butyl 4-[4-(4-tert-butylphenyl)-1H-imidazol-2-yl]piperidine-1-carboxylate;
-1-benzyl-3-[4-(4-fluorophenyl)-1H-imidazol-2-yl]piperidine;
-butyl 3-[4-(4-fluorophenyl)-1H-imidazol-2-yl]piperidine-1-carboxylate;
-2-[4-(4-fluorophenyl)-1H-imidazol-2-yl]piperidine;
-1-benzyl-2-[4-(4-fluorophenyl)-1H-imidazol-2-yl]piperidine;
-butyl 2-[4-(4-fluorophenyl)-1H-imidazol-2-yl]piperidine-1-carboxylate;
-4-[4-(1,1′-biphenyl-4-yl)-1H-imidazol-2-yl]-1-hexylpiperidine;
-butyl 4-[4-(1,1′-biphenyl-4-yl)-1H-imidazol-2-yl]piperidine-1-carboxylate;
-2-[4-(1,1′-biphenyl-4-yl)-1H-imidazol-2-yl]piperidine;
-3-[4-(1,1′-biphenyl-4-yl)-1H-imidazol-2-yl]piperidine;
-butyl 2-[4-(1,1′-biphenyl-4-yl)-1H-imidazol-2-yl]piperidine-1-carboxylate;
-butyl 3-[4-(1,1′-biphenyl-4-yl)-1H-imidazol-2-yl]piperidine-1-carboxylate;
-4-[4-(4-methoxyphenyl)-1H-imidazol-2-yl]piperidine;
-butyl 4-[4-(4-methoxyphenyl)-1H-imidazol-2-yl]piperidine-1-carboxylate;
and the pharmaceutically acceptable salts of the latter.

5. Use according to claim 4, characterized in that the compound of general formula (I) is chosen from the following compounds:
-butyl 4-[4-(4′-bromo-1,1′-biphenyl-4-yl)-1H-imidazol-2-yl]piperidine-1-carboxylate;
-tert-butyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]piperidine;
-1-benzyl-4-[4-(1,1′-biphenyl-4-yl)-1H-imidazol-2-yl]piperidine;
-1-(cyclohexylmethyl)-4-[4-(4-fluorophenyl)-1H-imidazol-2-yl]piperidine;
-4-[4-(1,1′-biphenyl-4-yl)-1H-imidazol-2-yl]-N-butylpiperidine-1-carboxamide;
-4-[4-(1,1′-biphenyl-4-yl)-1H-imidazol-2-yl]-N-butylpiperidine-1-carbothioamide;
-ethyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]-1-pentanoylpiperidine;
-1-benzyl-4-[4-(1,1′-biphenyl-4-yl)-1H-imidazol-2-yl]piperidine;
-butyl 4-[4-(4-tert-butylphenyl)-1H-imidazol-2-yl]piperidine-1-carboxylate;
-1-benzyl-3-[4-(4-fluorophenyl)-1H-imidazol-2-yl]piperidine;
-1-benzyl-2-[4-(4-fluorophenyl)-1H-imidazol-2-yl]piperidine;
-4-[4-(1,1′-biphenyl-4-yl)-1H-imidazol-2-yl]-1-hexylpiperidine;
-butyl 4-[4-(1,1′-biphenyl-4-yl)-1H-imidazol-2-yl]piperidine-1-carboxylate;
-2-[4-(1,1′-biphenyl-4-yl)-1H-imidazol-2-yl]piperidine;
-3-[4-(1,1′-biphenyl-4-yl)-1H-imidazol-2-yl]piperidine;
-butyl 2-[4-(1,1′-biphenyl-4-yl)-1H-imidazol-2-yl]piperidine-1-carboxylate;
-butyl 3-[4-(1,1′-biphenyl-4-yl)-1H-imidazol-2-yl]piperidine-1-carboxylate;
-butyl 4-[4-(4-methoxyphenyl)-1H-imidazol-2-yl]piperidine-1-carboxylate;
and the pharmaceutically acceptable salts of the latter.

6. Use of a compound of general formula (I) as defined in one of claims 1 to 5 or of a pharmaceutically acceptable salt of such a compound for preparing a medicament intended to treat pain.

7. Use according to claim 6, characterized in that the medicament prepared is intended to treat neuropathic pains and migraine.

8. As a medicament, a compound of general formula (I)_{M} 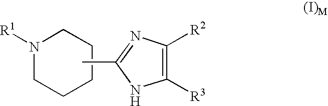
in racemic or enantiomeric form or any combination of these forms, in which:
R¹ represents a hydrogen atom or an —X—Y—R⁴ radical in which —X—Y— represents a bond, a —CO—, —CO—O—, —CO—NH— or —CS—NH— radical and R⁴ represents an alkyl, haloalkyl, cycloalkyl, cycloalkylalkyl, aryl or aralkyl radical, said aralkyl or aryl radicals being optionally substituted 1 to 4 times on the aryl radical by a radical or radicals chosen from a halogen atom and an alkyl, hydroxy or alkoxy radical;
R² represents an alkyl, cycloalkyl, cycloalkylalkyl, or aryl radical, said aryl being optionally substituted 1 to 4 times by a radical or radicals chosen from a halogen atom and an alkyl, haloalkyl, hydroxy, alkoxy, alkylthio, cyano, nitro, NR⁵R⁶, SO₂R⁷, arylamino or aryl radical, said arylamino or aryl being optionally substituted 1 to 4 times on the aryl group by a radical or radicals chosen from a halogen atom and an alkyl, hydroxy, alkoxy or alkylthio radical,
R⁵ and R⁶ representing independently a hydrogen atom or an alkyl radical or R⁵ and R⁶ forming together with the nitrogen atom already present a heterocycle with 5 to 7 members the additional members of which are chosen from —CH₂—, —O—, —S— and —NR⁸—,
R⁷ representing independently each time it is involved an alkyl radical,
R⁸ representing independently each time it is involved a hydrogen atom or an alkyl, haloalkyl, cycloalkyl, cycloalkylalkyl, aryl or aralkyl radical, said aralkyl or aryl radicals being optionally substituted 1 to 4 times on the aryl radical by a radical or radicals chosen from a halogen atom and an alkyl, hydroxy or alkoxy radical,
or R² represents the 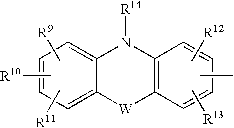
radical in which R⁹, R¹⁰, R¹¹, R¹² and R¹³ represent, independently, a hydrogen atom, a halogen, the OH group or an alkyl or alkoxy radical,
R¹⁴ represents a hydrogen atom or an alkyl radical,
and W does not exist, or represents a bond, or —O—, —S— or —NR¹⁵—, in which R¹⁵ represents a hydrogen atom or an alkyl radical,
or also R² represents the 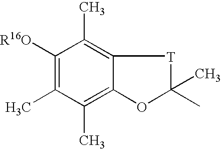
radical in which R¹⁶ represents a hydrogen atom or an alkyl radical,
and T represents a —(CH₂)ₘ— radical with m=1 or 2,
or finally R² represents the 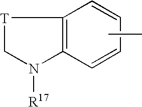
radical in which R¹⁷ represents a hydrogen atom or an alkyl, —NR¹⁸R¹⁹ or —Σ—CHR²⁰R²¹ radical,
Σ representing a linear or branched alkylene radical containing 1 to 6 carbon atoms,
R¹⁸ and R¹⁹ representing, independently, a hydrogen atom or an alkyl radical,
R²⁰ and R²¹ representing, independently, a hydrogen atom or a carbocyclic or heterocyclic aryl radical optionally substituted 1 to 4 times by one or more substituents chosen independently from the alkyl, OH, halogen, nitro, alkoxy and NR²²R²³ radicals,
R²² and R²³ representing, independently, a hydrogen atom or an alkyl radical,
and T represents a —(CH₂)ₘ— radical with m=1 or 2; and
R³ represents a hydrogen atom or an alkyl, cycloalkyl, cycloalkylalkyl, or aryl radical, said aryl being optionally substituted 1 to 4 times by a radical or radicals chosen from a halogen atom and an alkyl, haloalkyl, hydroxy, alkoxy, alkylthio, cyano, nitro, NR²⁴R²⁵, SO₂R²⁶ or aryl radical optionally substituted 1 to 4 times by a radical or radicals chosen from a halogen atom and an alkyl, hydroxy, alkoxy or alkylthio radical,
R²⁴ and R²⁵ representing independently a hydrogen atom or an alkyl radical or R²⁴ and R²⁵ forming together with the nitrogen atom already present a heterocycle with 5 to 7 members the additional members of which are chosen from —CH₂—, —O—, —S— and —NR²⁸—,
R²⁶ representing independently each time it is involved an alkyl radical,
and R²⁸ representing independently each time it is involved a hydrogen atom or an alkyl radical;
it being understood however that when R² does not represent one of the 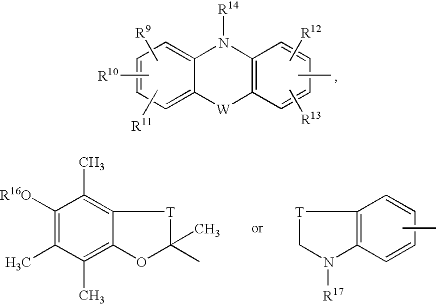
radicals, then R¹ does not represent a radical chosen from a hydrogen atom, an alkyl radical, an alkoxy radical or an optionally substituted aryl radical;
or the pharmaceutically acceptable salts of the compounds of general formula (I)_{M}.

9. Medicament characterized in that it is one of the following compounds:
-butyl 4-[4-(4′-bromo-1,1′-biphenyl-4-yl)-1H-imidazol-2-yl]piperidine-1-carboxylate;
-tert-butyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]piperidine;
-1-benzyl-4-[4-(1,1′-biphenyl-4-yl)-1H-imidazol-2-yl]piperidine;
-1-(cyclohexylmethyl)-4-[4-(4-fluorophenyl)-1H-imidazol-2-yl]piperidine;
-4-[4-(1,1′-biphenyl-4-yl)-1H-imidazol-2-yl]-N-butylpiperidine-1-carboxamide;
-4-[4-(1,1′-biphenyl-4-yl)-1H-imidazol-2-yl]-N-butylpiperidine-1-carbothioamide;
-ethyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]-1-pentanoylpiperidine;
-tert-butyl 4-[4-(4-fluorophenyl)-1H-imidazol-2-yl]piperidine-1-carboxylate;
-4-[4-(4-fluorophenyl)-1H-imidazol-2-yl]piperidine;
-1-benzyl-4-[4-(1,1′-biphenyl-4-yl)-1H-imidazol-2-yl]piperidine;
-butyl 4-[4-(4-tert-butylphenyl)-1H-imidazol-2-yl]piperidine-1-carboxylate;
-1-benzyl-3-[4-(4-fluorophenyl)-1H-imidazol-2-yl]piperidine;
-butyl 3-[4-(4-fluorophenyl)-1H-imidazol-2-yl]piperidine-1-carboxylate;
-2-[4-(4-fluorophenyl)-1H-imidazol-2-yl]piperidine;
-1-benzyl-2-[4-(4-fluorophenyl)-1H-imidazol-2-yl]piperidine;
-butyl 2-[4-(4-fluorophenyl)-1H-imidazol-2-yl]piperidine-1-carboxylate;
-4-[4-(1,1′-biphenyl-4-yl)-1H-imidazol-2-yl]-1-hexylpiperidine;
-butyl 4-[4-(1,1′-biphenyl-4-yl)-1H-imidazol-2-yl]piperidine-1-carboxylate;
-2-[4-(1,1′-biphenyl-4-yl)-1H-imidazol-2-yl]piperidine;
-3-[4-(1,1′-biphenyl-4-yl)-1H-imidazol-2-yl]piperidine;
-butyl 2-[4-(1,1′-biphenyl-4-yl)-1H-imidazol-2-yl]piperidine-1-carboxylate;
-butyl 3-[4-(1,1′-biphenyl-4-yl)-1H-imidazol-2-yl]piperidine-1-carboxylate;
-4-[4-(4-methoxyphenyl)-1H-imidazol-2-yl]piperidine;
-butyl 4-[4-(4-methoxyphenyl)-1H-imidazol-2-yl]piperidine-1-carboxylate;
or a pharmaceutically acceptable salt of one of the latter.

10. Product characterized in that it is a compound of general formula (I)_{M} as defined in claim 8 or a salt of such compound.

11. Product characterized in that it is one of the following compounds:
-butyl 4-[4-(4′-bromo-1,1′-biphenyl-4-yl)-1H-imidazol-2-yl]piperidine-1-carboxylate;
-tert-butyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]piperidine;
-1-benzyl-4-[4-(1,1′-biphenyl-4-yl)-1H-imidazol-2-yl]piperidine;
-1-(cyclohexylmethyl)-4-[4-(4-fluorophenyl)-1H-imidazol-2-yl]piperidine;
-4-[4-(1,1′-biphenyl-4-yl)-1H-imidazol-2-yl]-N-butylpiperidine-1-carboxamide;
-4-[4-(1,1′-biphenyl-4-yl)-1H-imidazol-2-yl]-N-butylpiperidine-1-carbothioamide;
-ethyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]-1-pentanoylpiperidine;
-tert-butyl 4-[4-(4-fluorophenyl)-1H-imidazol-2-yl]piperidine-1-carboxylate;
-4-[4-(4-fluorophenyl)-1H-imidazol-2-yl]piperidine;
-1-benzyl-4-[4-(1,1′-biphenyl-4-yl)-1H-imidazol-2-yl]piperidine;
-butyl 4-[4-(4-tert-butylphenyl)-1H-imidazol-2-yl]piperidine-1-carboxylate;
-1-benzyl-3-[4-(4-fluorophenyl)-1H-imidazol-2-yl]piperidine;
-butyl 3-[4-(4-fluorophenyl)-1H-imidazol-2-yl]piperidine-1-carboxylate;
-2-[4-(4-fluorophenyl)-1H-imidazol-2-yl]piperidine;
-1-benzyl-2-[4-(4-fluorophenyl)-1H-imidazol-2-yl]piperidine;
-butyl 2-[4-(4-fluorophenyl)-1H-imidazol-2-yl]piperidine-1-carboxylate;
-4-[4-(1,1′-biphenyl-4-yl)-1H-imidazol-2-yl]-1-hexylpiperidine;
-butyl 4-[4-(1,1′-biphenyl-4-yl)-1H-imidazol-2-yl]piperidine-1-carboxylate;
-2-[4-(1,1′-biphenyl-4-yl)-1H-imidazol-2-yl]piperidine;
-3-[4-(1,1′-biphenyl-4-yl)-1H-imidazol-2-yl]piperidine;
-butyl 2-[4-(1,1′-biphenyl-4-yl)-1H-imidazol-2-yl]piperidine-1-carboxylate;
-butyl 3-[4-(1,1′-biphenyl-4-yl)-1H-imidazol-2-yl]piperidine-1-carboxylate;
-4-[4-(4-methoxyphenyl)-1H-imidazol-2-yl]piperidine;
-butyl 4-[4-(4-methoxyphenyl)-1H-imidazol-2-yl]piperidine-1-carboxylate;
or a salt of one of the latter.

12. Pharmaceutical composition comprising, as active ingredient, at least one compound of general formula (I)_{M} as defined in claim 8 or a pharmaceutically acceptable salt of such compound.

13. Pharmaceutical composition comprising, as active ingredient, at least one of the following compounds:
-butyl 4-[4-(4′-bromo-1,1′-biphenyl-4-yl)-1H-imidazol-2-yl]piperidine-1-carboxylate;
-tert-butyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]piperidine;
-1-benzyl-4-[4-(1,1′-biphenyl-4-yl)-1H-imidazol-2-yl]piperidine;
-1-(cyclohexylmethyl)-4-[4-(4-fluorophenyl)-1H-imidazol-2-yl]piperidine;
-4-[4-(1,1′-biphenyl-4-yl)-1H-imidazol-2-yl]-N-butylpiperidine-1-carboxamide;
-4-[4-(1,1′-biphenyl-4-yl)-1H-imidazol-2-yl]-N-butylpiperidine-1-carbothioamide;
-ethyl 4-[4-(1,1′-biphenyl-4-yl)-1H-imidazol-2-yl]piperidine-1-carboxylate;
-4-[4-(1,1′-biphenyl-4-yl)-1H-imidazol-2-yl]-1-pentanoylpiperidine;
-tert-butyl 4-[4-(4-fluorophenyl)-1H-imidazol-2-yl]piperidine-1-carboxylate;
-4-[4-(4-fluorophenyl)-1H-imidazol-2-yl]piperidine;
-1-benzyl-4-[4-(1,1′-biphenyl-4-yl)-1H-imidazol-2-yl]piperidine;
-butyl 4-[4-(4-tert-butylphenyl)-1H-imidazol-2-yl]piperidine-1-carboxylate;
-1-benzyl-3-[4-(4-fluorophenyl)-1H-imidazol-2-yl]piperidine;
-butyl 3-[4-(4-fluorophenyl)-1H-imidazol-2-yl]piperidine-1-carboxylate;
-2-[4-(4-fluorophenyl)-1H-imidazol-2-yl]piperidine;
-1-benzyl-2-[4-(4-fluorophenyl)-1H-imidazol-2-yl]piperidine;
-butyl 2-[4-(4-fluorophenyl)-1H-imidazol-2-yl]piperidine-1-carboxylate;
-4-[4-(1,1′-biphenyl-4-yl)-1H-imidazol-2-yl]-1-hexylpiperidine;
-butyl 4-[4-(1,1′-biphenyl-4-yl)-1H-imidazol-2-yl]piperidine-1-carboxylate;
-2-[4-(1,1′-biphenyl-4-yl)-1H-imidazol-2-yl]piperidine;
-3-[4-(1,1′-biphenyl-4-yl)-1H-imidazol-2-yl]piperidine;
-butyl 2-[4-(1,1′-biphenyl-4-yl)-1H-imidazol-2-yl]piperidine-1-carboxylate;
-butyl 3-[4-(1,1′-biphenyl-4-yl)-1H-imidazol-2-yl]piperidine-1-carboxylate;
-4-[4-(4-methoxyphenyl)-1H-imidazol-2-yl]piperidine;
-butyl 4-[4-(4-methoxyphenyl)-1H-imidazol-2-yl]piperidine-1-carboxylate;
or a pharmaceutically acceptable salt of one of the latter.
